(19) United States
(12) Patent Application Publication
(10) Pub. No.: US 2011/0301137 A1
(43) Pub. Date: Dec. 8, 2011

(54) DERMACEUTICAL GEL MADE USING SODIUM FUSIDATE AND A PROCESS TO MAKE IT

(75) Inventors: Vanangamudi Subramaniam Sulur, Chennai (IN); Madhavan Srinivasan, Chennai (IN); Neelakandan Narayanan Chulliel, Chennai (IN); Haridas Sankar, Mumbai (IN); Kausik Ghosh, Chennai (IN)

(73) Assignee: APEX LABORATORIES PRIVATE LIMITED, CHENNAI, TN (IN)

(21) Appl. No.: 13/201,625

(22) PCT Filed: Feb. 16, 2010
(86) PCT No.: PCT/IB2010/050684

§ 371 Date: Aug. 16, 2011

(30) Foreign Application Priority Data
Feb. 18, 2009 (IN) 356/MUM/2009

(51) Int. Cl. A61K 31/575; A61P 17/00

(52) U.S. Cl. 514/182

(57) ABSTRACT

The invention discloses a process to make dermaceutical gel containing Fusidic acid which is formed in situ from Sodium Fusidate as the starting raw material, wherein Sodium Fusidate is converted into Fusidic acid under oxygen-free environment comprising an inert gas, preferably nitrogen. The gel produced by the process of the present invention has greater shelf-life stability and the finer particle size of the API than the conventional creams containing Fusidic acid. The gel also contains Fusidic acid as the API that has been formed in situ from Sodium Fusidate, in a gel base; said gel base comprising a natural, semi-synthetic or synthetic polymers, a preservative, an acid, an alkali, a co-solvent, along with water, preferably purified water. The gel produced by the process of the present invention further optionally contains an ingredient selected from a group comprising, an anti oxidant, a chelating agent, and a humectant, or any combination thereof.

### FIELD OF INVENTION

The present invention relates to primary and secondary bacterial skin infections and in particular it relates to the process of making a gel useful in the treatment of these infections, said gel incorporating Fusidic acid that has been created in situ using Sodium Fusidate as the starting Active Pharmaceutical Ingredient (API).

### BACKGROUND OF INVENTION

Numerous treatments, both topical and systemic, are available for the primary and secondary skin infection caused by sensitive Gram +ve organisms such as Staphylococcus aureus, Streptococcus spp etc. Topical and systemic bacterial infection treatment compositions typically employ at least one active pharmaceutical ingredient (API) in combination with a base component. In the cream form, the APIs typically comprise an antibiotic/antibacterial such as Fusidic acid and the like.

Fusidic acid is available in cream and ointment forms. In the currently available Fusidic acid creams, Fusidic acid in fine powder form is used as source API. The small particle size enhances its dermal contact by providing a large specific surface area and penetration, and provides a smooth feel on application to skin. However, a serious shortcoming of the fine size of Fusidic acid particles is that it presents an enormous surface area for contact and reaction with molecular Oxygen during manufacture, handling, and processing of the cream. This has serious implications to its chemical stability and results in rapid reduction in potency of the API (Fusidic acid) in the final cream formulation.

Degradation due to oxidation is a major cause of instability of currently available Fusidic acid creams. Table 1 show that the degradation in the API samples (Fusidic acid) exposed to oxygen ranged between 7.7% and 11% for conditions ranging from room temperature to 45° C. when analysed at three months of exposure period at the above conditions.

It is known that greater the exposure time of Fusidic acid as the raw API to Oxygen, greater the limitations on stabilising Fusidic acid in a formulation. However, there is no published data on the stability of Fusidic acid over a period of time.

As an alternative to Fusidic acid, Sodium Fusidate is known to have been used to make dermaceutical medicaments for topical application. However, these are in the form of ointment rather than cream. Drawbacks of ointments over creams are well known and it's generally preferable to use creams rather than ointments for topical application. It is also preferable to use gel forms over creams or ointments.

Several aspects of Fusidic acid as an API are known:
It is thermolabile
It is available in cream formulations
It can be obtained from Sodium Fusidate by dissolving the latter in an aqueous phase and adding acid to the solution, whereby Fusidic acid precipitates. However, the Fusidic acid precipitate is difficult to process into a gel form first due to its coarse and uneven particle size and second retrieving Fusidic acid from wet cake involves drying and further handling which deteriorates the Fusidic acid due to exposure to oxygen
The stability of the API in a Fusidic acid gel is unreliable due to the thermolabile nature of Fusidic acid

Stabilization of medicaments containing Fusidic acid against oxidation involves observing a number of stringent precautionary procedures during manufacture and storage. These include:
replacing Oxygen in pharmaceutical containers with inert gases such as Nitrogen, Carbon dioxide, Helium and the like
avoiding contact of the medicament with heavy metal ions which catalyze oxidation,
storing the API at reduced temperatures throughout its shelf life before processing

In practice this means stricter controls during the manufacture as well as storage of such API (storing it typically at 2° C. to 8° C. in air-tight containers throughout their shelf life).

Furthermore, there are currently no gels available that use a stable form of Fusidic acid. Gels provide some advantages over creams and ointment both in terms of applicability and removal.

There is therefore a need to provide a gel for topical treatment that uses stable form of Fusidic acid.

There is also a need to provide a process of making a Fusidic acid gel in which Fusidic acid will be of greater stability than the stability of the Fusidic acid in the conventionally available tropical treatment compositions such as creams and ointments, particularly at the time of their manufacture, and which will sustain its stability at an acceptable level throughout the gel's shelf life.

### Objects and Advantages of Invention

Accordingly, one of the objects of the present invention is to provide a gel containing stable form of fusidic acid.

Another object of the present invention is to provide a process of making a pharmaceutically acceptable gel which contains Fusidic acid as the active API but which has greater stability of the API than the Fusidic acid manufactured using other means, throughout the gel's shelf life.

### BRIEF SUMMARY OF INVENTION

The invention discloses a process to make dermaceutical gel containing Fusidic acid which is formed in situ from Sodium Fusidate as the starting raw material, wherein Sodium Fusidate is converted into Fusidic acid under oxygen-free environment comprising an inert gas, preferably nitrogen. The gel produced by the process of the present invention has greater shelf-life stability and the finer particle size of the API than the conventional creams containing Fusidic acid. The gel produced by the process of the present invention contains Fusidic acid as the API that has been formed in situ from Sodium Fusidate, in a gel base; said gel base comprising a natural, semi-synthetic or synthetic polymers, a preservative, an acid, an alkali, a co-solvent, along with water, preferably purified water. The gel produced by the process of the present invention further optionally contains an ingredient selected from a group comprising, an anti oxidant, a chelating agent, and a humectant, or any combination thereof.

### DETAILED DESCRIPTION OF INVENTION

We discussed earlier the known aspects of the topical preparations that have Fusidic acid and Sodium Fusidate as the APIs. It is evident from the current state of knowledge that:
Gels containing Fusidic acid that is made using Sodium Fusidate as starting API are not available.
There is no published data on the stability of Sodium Fusidate as the API.
Sodium Fusidate is not considered to be inherently more stable as an API than Fusidic acid.

In the face of this, it has been surprisingly discovered that Sodium Fusidate as an API is significantly more stable than Fusidic acid and that Fusidic acid deteriorates more rapidly than Sodium Fusidate.

There is no published data on the stability of Sodium Fusidate as the API. The applicant carried out experiments on Sodium Fusidate to evaluate its stability. It can be seen from Table 2 that the degradation of Sodium Fusidate over a temperature range of room temperature to 45° C. ranged between 2.45% and 6%.

Tables 1 and 2 also show the comparison between the stability of the Fusidic acid and Sodium Fusidate as raw APIs. The study was carried out using an in-house HPLC method developed by the applicant, which the applicant believes is a true stability-indicating method as opposed to the titration method suggested in British Pharmacopoeia (BP). This is because the BP method does not differentiate between the intact API and the degraded form.

### Stability Analysis of Fusidic Acid:

TABLE 1
Results Of 3-Month-Old Fusidic Acid (API) Analysis By Stability
Indicating HPLC Method And Titration Method

| | | | Fusidic Acid Assay | | Percentage Drop | | |
|---|---|---|---|---|---|---|---|
| | Condi- | *Initial | (%) | | (%) | | |
| S.No | tions | (%) | Titration | HPLC | Titration | HPLC | Remarks |
| 1 | RT | 100.6 | 99.21 | 92.93 | 1.39 | 7.67 | API |
| | (Open) | | | | | | ana- |
| 2 | RT | | 99.02 | 94.37 | 1.58 | 6.23 | lysed |
| | (Closed) | | | | | | After 3 |
| 3 | 45° C. | | 98.52 | 89.52 | 2.08 | 11.08 | Months |
| | (Open) | | | | | | |
| 4 | 45° C. | | 99.10 | 92.12 | 1.50 | 8.48 | |
| | (Closed) | | | | | | |

Name of the Sample: Fusidic Acid BP Pack: Open & Closed Petri dish

### Stability Analysis of Sodium Fusidate:

TABLE 2
Results Of 3 Months Old Sodium Fusidate (API) Analysis By
Stability Indicating HPLC Method And Titration Method

| | | | Sodium Fusidate | | | | |
|---|---|---|---|---|---|---|---|
| | Condi- | *Initial | Assay(%) | | Percentage (%) | | |
| S.No | tions | (%) | Titration | HPLC | Titration | HPLC | Remarks |
| 1 | RT | 98.7 | 97.71 | 96.25 | 0.99 | 2.45 | API |
| | (Open) | | | | | | ana- |
| 2 | RT | | 98.85 | 97.67 | −0.15 | 1.03 | lysed |
| | (Closed) | | | | | | After 3 |
| 3 | 45° C. | | 97.07 | 92.65 | 1.63 | 6.05 | Months |
| | (Open) | | | | | | |
| 4 | 45° C. | | 97.16 | 92.96 | 1.54 | 5.74 | |
| | (Closed) | | | | | | |

Name of the Sample: Sodium Fusidate BP Pack: Open & Closed Petri dish

In both studies the * Initial denotes the results of the samples tested at the time of receipt of the API from the supplier.

It can be observed from Tables 1 and 2 that:
In the case of Fusidic Acid, there is about 7.7% loss in 3 Months at room temperature (open condition) and about 11% loss in 3 Months at 45° C. (open condition).
In the case of Sodium Fusidate, there is about 2.5% loss in 3 Months at room temperature (open condition) and about 6% loss in 3 Months at 45° C. (open condition).

The data thus shows that Sodium Fusidate as an API is more stable than Fusidic acid.

The applicants explored the possibility of making a gel (rather than a cream or an ointment) using Sodium Fusidate (rather than Fusidic acid). Although Sodium Fusidate has been used in dermaceutical applications, it has not been possible to make creams or gels that use Sodium Fusidate. This is because of the inherent alkalinity of Sodium Fusidate (pH 7.5 to 9), which means it cannot be used in a cream or gel form therefore all products manufactured using Sodium Fusidate as starting material are ointments. A dermaceutical gel that uses Sodium Fusidate would exploit the benefit of the fact that Sodium Fusidate is more stable than Fusidic acid and it would also provide a gel formulation which is far superior in its application qualities than an ointment and creams. It would thus fill an existing need for a composition that has better stability than currently available creams containing Fusidic acid.

The applicant therefore surprisingly discovered that in order to achieve greater stability of the API in a dermaceutical composition, Sodium Fusidate rather than Fusidic acid may be used as the starting API during the gel's manufacture. Using Sodium Fusidate as starting material eliminates the drawback associated with the manufacture and storage of existing Fusidic acid compositions.

The applicant has also discovered that the Fusidic acid gel prepared using Sodium Fusidate as the staring API shows good chemical stability, efficacy, and microbial sensitivity.

The application discloses a process of making a pharmaceutical gel containing Fusidic acid (the API) that has been prepared using Sodium Fusidate as the starting API, in which Fusidic acid forms in-situ under totally oxygen free environment (created using an inert gas, preferably nitrogen) by slow addition of an acid, into a molecular dispersion form (due to the presence of a co-solvent) at the intermediate stage, and which Fusidic acid regenerates as an extremely fine dispersion when added to a final gel base, thereby resulting in a finely and homogeneously dispersed Fusidic acid in the final gel. All these operations are performed in an environment free of atmospheric oxygen in an environment created using inert gas, preferably nitrogen. The gel made using the process of the present invention contains Fusidic acid as the API that has been formed in situ from Sodium Fusidate, in a gel base comprising, a preservative, an acid, an alkali, a co-solvent, a natural, semisynthetic, or synthetic polymer, a chelating agent, a humectant, an antioxidant along with water, preferably purified water.

The APIs which may be employed in the process of the present invention as starting APIs are either acid-based actives or their salts well known in the art of treating bacterial primary and secondary infections. Examples of suitable acid-based actives or their salts which may be used include, but are not limited to Sodium Fusidate.

These acid-based active compounds or their salts require a base component to be used in the pharmaceutical composition that uses the compounds, since the compounds cannot, by themselves, be deposited directly on to human skin due to their harshness.

The gel base made using the process of the present invention optionally further comprises an ingredient selected from a group comprising an anti oxidant, a chelating agent, and a humectant, or any combination thereof.

The present invention also provides a process to make a novel gel that has been produced using Sodium Fusidate as the starting raw material, and which gel contains Fusidic acid of high therapeutic efficacy and of chemical stability that is generally superior to the commercially available creams containing Fusidic acid.

The Fusidic acid gel made using the process of the present invention has been manufactured in a totally oxygen free environment under purging with inert gas, preferably nitrogen, and applying vacuum. Under these conditions, the Sodium Fusidate is converted in situ into Fusidic acid. The gel of the present invention is used in the treatment of bacterial skin infections.

The preferred embodiments and the accompanying embodiments describe the gel of the present invention and the process of making it.

### Preferred Embodiment No. 1

The preferred embodiment of the invention discloses a process to make a dermaceutical gel containing Fusidic acid, said process comprising the step of using sodium fusidate as the raw API and converting it in situ into Fusidic acid under oxygen-free environment in a gel base.

### Embodiment No. 1

In an embodiment of the present invention the process of making the composition is disclosed, wherein the said gel base of the preferred embodiment no. 1 comprises, a preservative, an acid, an alkali, a co-solvent, a natural, semisynthetic, or synthetic polymer, along with water, preferably purified water, and wherein said step of converting the sodium fusidate in situ into Fusidic acid comprises the steps of:
a. heating water, said water being preferably purified water, preferably 10 to 75% w/w, more preferably 57% w/w in a mixing vessel to 50° C. to 60° C.,
b. dissolving 0.05 to 0.5% w/w preservative, more preferably 0.2% w/w Benzoic Acid, in the said mixing vessel,
c. adding a polymer, said polymer being preferably a natural, semisynthetic, or synthetic polymer, preferably 1 to 5% w/w, more preferably 1.25% w/w Carbomer 934 P to said mixing vessel and thoroughly mixing using said agitator at 10 to 50 RPM and homogenizing the mixture at 1000 to 3000 RPM and under vacuum of minus 1000 to minus 300 mm of mercury,
d. cooling the mixture in said mixing vessel to 40° C. preferably by circulating cold water at a temperature of 8 to 15° C. from cooling tower in the jacket of the mixing vessel,
e. adding in an API-vessel a co-solvent, selected from a group comprising propylene glycol, hexylene glycol, polyethylene glycol-400, and the like, preferably propylene glycol, preferably 5 to 50% w/w, more preferably 25% w/w,
f. adding to said API-vessel of step e sodium fusidate in an amount between 1 to 5% w/w, more preferably 2.08% w/w and dissolving using mechanical stirrer at 1000 to 3000 RPM under an inert gas flushing, said inert gas being preferably nitrogen,
g. adjusting the pH of the mixture in the API-vessel to below 2 by adding acid, preferably 0.005 to 0.5% w/w, more preferably 4% w/w of 1 Molar Nitric acid solution,
h. transferring the contents of said API-vessel to the mixing vessel of step d with continuous stirring at 10 to 50 RPM and homogenizing the mixture at 1000 to 3000 RPM under an inert gas flushing and under vacuum of minus 1000 to minus 300 mm of mercury, said inert gas being preferably nitrogen,
i. cooling the contents of the mixing vessel of step h to 30° C. to 35° C. using circulation of chilled water from cooling tower at a temperature of 8° C. to 15° C. into the jacket of said mixing vessel,
j. adjusting the pH of the contents in mixing vessel of step i between 3.5 and 6.0 by adding preferably 0.1 to 5% w/w alkalizing agent, more preferably 0.4% w/w Triethanolamine and mixing at 10 to 50 RPM under an inert gas flushing and under vacuum of minus 1000 to minus 300 mm of mercury, said inert gas being preferably nitrogen,
k. turning off the agitator and removing the contents of the mixing vessel of step j to a storage container.

### Embodiment No. 2

In an embodiment of the present invention, the co-solvent also serves as a humectant. However, in another embodiment of the invention, an additional humectant may be added to the mixing vessel of step a in embodiment no. 1, selected from a group comprising Glycerin, Sorbitol, Propylene glycol and the like, either singly or any combination thereof, to form a proportion from about 1% (w/w) to 30% (w/w), preferably 20% (w/w), more preferably 10% (w/w) of Propylene glycol.

### Embodiment No. 3

In another embodiment of the present invention the process described in embodiment no. 2 further incorporates adding and dissolving a chelating agent, to the mixing vessel of step a in embodiment no. 1 selected from a group comprising Disodium EDTA and the like, either singly or any combination thereof, to form a proportion from about 0.001% (w/w) to 1% (w/w), preferably 0.05% (w/w), more preferably 0.01% (w/w) of Disodium EDTA.

### Embodiment No. 4

In a further embodiment of the present invention the process described in embodiments no. 2 to 3 further incorporate an anti oxidants, added and dissolved in step e of embodiment no. 1 selected from a group comprising Butylated Hydroxy Anisole, Butylated Hydroxy Toluene and the like from about 0.001% (w/w) to 5% (w/w), preferably 0.1% (w/w), more preferably 0.01% (w/w) of Butylated Hydroxy Toluene.

### Embodiment No. 5

In a further embodiment of the present invention the process described in embodiments no. 2 to 3 further incorporates a buffering agent, added after step j of embodiment no. 1, said buffering agent being selected from a group comprising Di Sodium Hydrogen Ortho Phosphate, Sodium Hydrogen Ortho Phosphate and the like from about 0.01% (w/w) to 1.00% (w/w), preferably 0.5% (w/w), more preferably 0.05% (w/w).

### Embodiment No. 6

In an embodiment of the present invention the process of making the composition is disclosed, wherein the said gel base comprises, a preservative, an acid, an alkali, a co-solvent, an anti-oxidant, a chelating agent, a humectant, a natural, semisynthetic, or synthetic polymer, along with water, preferably purified water, and wherein said step of converting the sodium fusidate in situ into Fusidic acid comprises the steps of:
a. heating water, said water being preferably purified water, preferably 10 to 75% w/w, more preferably 57% w/w in a mixing vessel to 50° to 60° C., adding and dissolving 0.05 to 0.5% w/w preservative, more preferably 0.2% w/w Benzoic Acid, and 0.001 to 1 w/w Chelating Agent, more preferably 0.01% w/w Disodium Edetate by stirring using agitator at 10 to 50 RPM.
b. adding humectant selected from a group comprising propylene glycol, hexylene glycol, polyethylene glycol-400, and the like, preferably propylene glycol, preferably 1 to 15% w/w, more preferably 10% w/w to said mixing vessel and thoroughly mixing using an agitator at 10 to 50 RPM while maintaining the temperature of the mixture at 50° C. to 60° C.,
c. adding a polymer, said polymer being preferably a natural, semisynthetic, or synthetic polymer, preferably 1 to 5% w/w, more preferably 1.25% w/w Carbomer 934 P to said mixing vessel and thoroughly mixing using said agitator at 10 to 50 RPM and homogenizing the mixture at 1000 to 3000 RPM and under vacuum of minus 1000 to minus 300 mm of mercury,
d. cooling the mixture in said mixing vessel to 40° C. preferably by circulating cold water at a temperature of 8 to 15° C. from cooling tower in the jacket of the mixing vessel,
e. adding in an API-vessel a co-solvent, selected from a group comprising propylene glycol, hexylene glycol, polyethylene glycol-400, and the like, preferably propylene glycol, preferably 5 to 50% w/w, more preferably 25% w/w and adding & dissolving an anti-oxidant selected from a group comprising Butylated hydroxy anisole, Butylated hydroxy toluene and the like, preferably 0.01 to 0.1% w/w, more preferably 0.01 of Butylated Hydroxy Toluene,
f. adding in said API-vessel of step e sodium fusidate in an amount between 1 to 5% w/w, more preferably 2.08% w/w and dissolving using mechanical stirrer at 1000 to 3000 RPM under an inert gas flushing, said inert gas being preferably nitrogen,
g. adjusting the pH of the mixture in the API-vessel to below 2 by adding acid, preferably 0.005 to 0.5% w/w, more preferably 4% w/w of 1 Molar Nitric acid solution,
h. transferring the contents of said API-vessel to the mixing vessel of step d with continuous stirring at 10 to 50 RPM and homogenizing the mixture at 1000 to 3000 RPM under an inert gas flushing and under vacuum of −1000 to −300 mm of mercury, said inert gas being preferably nitrogen,
i. cooling the contents of the mixing vessel of step h to 30° C. to 35° C. using circulation of chilled water from cooling tower at 8° C. to 15° C. into the jacket of said mixing vessel,
j. adjusting the pH of the contents of said mixing vessel of step i between 3.5 and 6.0 by adding preferably 0.1 to 5% w/w alkalizing agent, more preferably 0.4% w/w Triethanolamine and mixing at 10 to 50 RPM under an inert gas flushing and under vacuum of −1000 to −300 mm of mercury, said inert gas being preferably nitrogen,
k. further adjusting the pH of the contents of said mixing vessel of step j by adding a buffering agent, buffering agent selected from a group comprising Di Sodium Hydrogen Ortho Phosphate, Sodium Hydrogen Ortho Phosphate and the like from about 0.01% (w/w) to 1.00% (w/w), preferably 0.5% (w/w), more preferably 0.05% (w/w),
l. turning off the agitator and removing the contents of said mixing vessel of step k to a storage container.

### Preferred Embodiment No. 2

A novel dermaceutical gel containing a gel base and Fusidic acid, said Fusidic acid being made in situ under oxygen-free environment using Sodium Fusidate, wherein said cream comprises Fusidic acid made in situ by a conversion of Sodium Fusidate, and said gel base comprising a natural, semi-synthetic or synthetic polymers, a preservative, an acid, an alkali, a co-solvent, along with water, preferably purified water.

### Embodiment No. 7

According to another embodiment of the present invention, there is provided a dermaceutical gel for the topical treatment of bacterial skin infections on human skin, wherein the composition of the gel as disclosed in the preferred embodiment no. 2 is:
from about 0.1% (w/w) to about 25% (w/w) by weight, preferably from about 0.5% to about 5% by weight and most preferably from about 1% (w/w) to 2% (w/w) by weight, of an acid form active compound, preferably sodium fusidate and,
a gel base containing natural or semisynthetic or synthetic polymers, co-solvents, acids, alkalis, buffering agents, preservatives, anti oxidants, chelating agents, humectants, water, all weights based on the weight of the composition, wherein
   natural polymers are selected from tragacanth, pectin, carrageen, agar, and alginic acid and Synthetic & semi-synthetic polymers are selected from methylcellulose, hydroxyethylcellulose, carboxymethylcellulose, hydroxypropylcellulose, hydroxypropylmethylcellulose and carbopols and the like from about 0.5% (w/w) to 10% (w/w),
   co-solvents are selected from a group comprising Propylene Glycol, Hexylene Glycol, PolyEthylene Glycol-400 and the like from about 5% (w/w) to 50% (w/w),
   acids such as HCl, H2So4, HNO3, Lactic acid and the like from about 0.005% (w/w) to 0.5% (w/w),
   adding an alkalizing agent, preferably 0.1 to 5% w/w, more preferably 0.4% w/w Triethanolamine,
   preservatives are selected from a group comprising Methylparaben, Propylparaben, Chlorocresol, Potassium sorbate, Benzoic acid and the like from about 0.05% (w/w) to 0.5% (w/w).

### Embodiment No. 8

According to another embodiment of the present invention, a gel is disclosed wherein the gel base of preferred embodiment no. 2 further comprises a buffering agent selected from a group comprising Di Sodium Hydrogen Ortho Phosphate, Sodium Hydrogen Ortho Phosphate and the like in a proportion from about 0.05% (w/w) to 1.00% (w/w).

### Embodiment No. 9

According to another embodiment of the present invention, a gel is disclosed wherein the gel base of embodiment no. 7 further comprises an anti-oxidant selected from a group comprising Butylated Hydroxy Anisole, Butylated Hydroxy Toluene and the like in a proportion from about 0.05% (w/w) to 5% (w/w).

### Embodiment No. 10

According to another embodiment of the present invention, a gel is disclosed wherein the gel base of preferred embodiment no. 7 and 8 further comprises a chelating agent selected from a group comprising Disodium EDTA and the like in a proportion from about 0.05% (w/w) to 1% (w/w).

### Embodiment No. 11

According to another embodiment of the present invention, a gel is disclosed wherein the gel base of preferred embodiment no. 7-9 further comprises a humectant selected from a group comprising Glycerin, Sorbitol, and the like in a proportion from about 5% (w/w) to 20% (w/w).

### Embodiment No. 12

According to another embodiment of the present invention, a method to treat primary and secondary skin infections is disclosed, said method comprising applying a gel as disclosed in any of embodiments 7 to 11 and the preferred embodiment no. 2.

### Embodiment No 13

According to another embodiment of the present invention, a method to treat primary and secondary skin infections is disclosed, said method comprising applying a gel made using the processes as disclosed in any of embodiments 2 to 6 and the preferred embodiment no. 1.

The gel obtained using the process of the present invention is homogenous and white to off white in colour and viscous in consistency. The pH of the product made using the process of the present invention is from about 3 to 6. On the other hand, Sodium Fusidate ointments that are commercially available are greasy and cosmetically non elegant.

It is essential that the active drug penetrates the skin for the optimum bio-dermal efficacy. The particle size of the active drug plays an important role here. It is necessary that the active drug is available in a finely dispersed form for the product to be being efficacious. Also this is to be achieved in the safe pH compatible environment of skin (4.0 to 6.0). To achieve all these, it is essential to choose proper vehicles or co-solvents for the dissolution or dispersion of the drug.

Particle size analysis was carried out on the cream made using the process of the present invention and on some commercially available product samples (samples A, C, D, F, G, and K). Maximum and minimum particle sizes, mean particle size and standard deviation and the coefficient of variation were assessed.

TABLE 3

| | Minimum | Maximum | Mean | | Coefficient |
|---|---|---|---|---|---|
| | Particle | Particle | Particle | Standard | of |
| | Size (μm) | Size (μm) | Size (μm) | Deviation | Variation |
| Invention | 2.41 | 15.39 | 9.95 | 3.995 | 0.401 |
| A | 7.23 | 39.58 | 18.09 | 9.251 | 0.511 |
| C | 6.07 | 32.69 | 14.11 | 6.692 | 0.474 |
| D | 9.8 | 27.52 | 18.48 | 4.98 | 0.269 |
| F | 7.93 | 19.90 | 14.82 | 4.033 | 0.272 |
| G | 7.29 | 29.48 | 15.25 | 6.065 | 0.398 |
| K | 5.75 | 32.63 | 16.80 | 8.112 | 0.483 |

The particle size distribution analysis clearly indicates the presence of Fusidic acid of fine particle size in the product of the present invention, the size that is advantageously much reduced than the conventional products. This is attributed to the fact that the instant product is made using Sodium Fusidate using in situ conversion of Sodium Fusidate to Fusidic acid in a finely dispersed form. All of the measured parameters are better than those found for the commercially available creams containing Fusidic acid. This is another clear advantage of the product disclosed herein over the commercially available products.

The product of the present invention is efficacious due to the pronounced antibacterial activity of the regenerated Fusidic acid which is available in reduced particle size than the conventional products, and in a finely dispersed form.

The inventor has screened different co-solvents such as Propylene Glycol, Hexylene Glycol, PolyEthyleneGlycol-400 & the like and dissolved the Sodium Fusidate in one of above co-solvents varying from about 5% (w/w) to 40% (w/w) under inert gas purging and under vacuum and converted to Fusidic acid in-situ by adding an acid such as HCl, H₂SO₄, HNO₃, Lactic acid and the like from about 0.005% (w/w) to about 0.5% (w/w) under stirring and obtained Fusidic acid in more stabilized and solution form, which makes our final product in a gel base which easily penetrates the skin and highly efficacious, and also highly derma compatible by having a pH of about 3.0 to about 6.0.

The stability of the product is confirmed by the stability studies performed for 6 months as per ICH guidelines and a comparison of stress studies done for in-house product with those on samples of commercially available comparable products.

### Experimental Data

API-stability experiments were carried out (see tables 4-9) using the product of the present invention and products currently commercially available. Tests were carried out to observe (or measure as appropriate) the physical appearance of the product, the pH value and assay of the API over a period of time. Tests were also carried out to assess the stability by subjecting the product to stress studies such as autoclave test and oxydative degradation test.

Further, in vitro antimicrobial zone of inhibition studies were also carried out over a period of time.

Each gram of product of the present invention used for the tests contained Sodium Fusidate in the amount required to produce 2% (w/w) Fusidic acid in the finished product.

The product used for the Stability Studies, Autoclave and Oxidative degradation tests contained approximately 10% extra API (overages). The product of the present invention used for studies contained Fusidic acid gel prepared using Sodium Fusidate as starting material. It was packaged in an aluminium collapsible tube and each gram of the product contained 20.8 mg of Sodium Fusidate (in conformance with BP), which is equivalent to 20 mg of Fusidic acid (BP conformant).

### Product: Sodium Fusidate Gel

PACK: Aluminum Collapsible tube; Composition: Each gm contains: Sodium

### Fusidate BP Equivalent to Fusidic Acid BP 2.0%

TABLE 4
Description Test, Batch No. SFG-09
Measured parameter: Physical appearance
Best value of measured parameter: Homogeneous White to off
White Viscous Gel
Method of measurement: Observation by naked eye

| | | 1st | 2nd | 3rd | 6th |
|---|---|---|---|---|---|
| Conditions | Initial | Month | Month | Month | Month |
| 40° C. 75% | Best value | Best | Best | Best | Best |
| RH | | value | value | value | value |
| 30° C. 65% | — | Do | Do | Do | Do |
| RH | | | | | |
| 25° C. 60% | — | Do | Do | Do | Do |
| RH | | | | | |
| Temperature | — | Do | — | — | — |
| cycling | | | | | |
| Freeze thaw | — | Do | — | — | — |

Note:
best value indicates that the measured physical appearance matched the best value

TABLE 5
pH Test, Batch No. SFG-09
Measured parameter: pH; Limits of measured parameter: 3-6
Method of measurement: Digital pH Meter

| | | 1st | 2nd | 3rd | 6th |
|---|---|---|---|---|---|
| Conditions | Initial | Month | Month | Month | Month |
| 40° C. 75% | 5.11 | 5.10 | 5.09 | 5.08 | 5.09 |
| RH | | | | | |
| 30° C. 65% | — | 5.09 | 5.08 | 5.08 | 5.10 |
| RH | | | | | |
| 25° C. 60% | — | 5.10 | 5.08 | 5.09 | 5.11 |
| RH | | | | | |
| Temperature | — | 5.08 | — | — | — |
| cycling | | | | | |
| Freeze thaw | — | 5.09 | — | — | — |

TABLE 6
Assay (%) Test, Batch No. SFG-09
Measured parameter: Assay (%); Limits of measured
parameter: 90-110
Method of measurement: HPLC Method

| | | 1st | 2nd | 3rd | 6th |
|---|---|---|---|---|---|
| Conditions | Initial | Month | Month | Month | Month |
| 40° C. 75% | 108.60 | 108.46 | 108.36 | 108.21 | 108.11 |
| RH | | | | | |
| 30° C. 65% | — | 108.50 | 108.46 | 108.36 | 108.21 |
| RH | | | | | |
| 25° C. 60% | — | 108.59 | 108.55 | 108.49 | 108.36 |
| RH | | | | | |
| Temp cycling | — | 107.13 | — | — | — |
| Freeze thaw | — | 107.25 | — | — | — |

It is apparent from tables 4-6 that on all counts, the pH value, the physical appearance, and stability, the product of the present invention is quite good.

Table 7 provides reference dates for samples A-I which were taken from commercially available creams of Fusidic acid and used for analysis.

TABLE 7

| | Mfg. | Exp. |
|---|---|---|
| Sample Number | Date | Date |
| Present invention | August 2009 | July 2011 |
| Sample A | August 2009 | July 2011 |
| Sample B | August 2009 | July 2011 |
| Sample C | July 2009 | June 2011 |
| Sample D | July 2009 | June 2011 |
| Sample E | August 2009 | July 2011 |
| Sample F | August 2009 | July 2011 |
| Sample G | August 2009 | July 2011 |
| Sample H | July 2009 | June 2011 |
| Sample I | December 2009 | November 2011 |

TABLE 8
Autoclave Analysis (%) Test,
Measured parameter: Assay (%); Limits of measured parameter: 90-110%
Method of measurement: HPLC Method

| | | | | | | | | Average drop |
|---|---|---|---|---|---|---|---|---|
| | | | | | | | | of |
| | Name of the | Analysis-I (%) | | | Analysis-II (%) | | | Analysis-I & |
| Sr. | Products and | | After | Drop in | | After | Drop in | Analysis-II |
| No | Details | Initial | Autoclave | % | Initial | Autoclave | % | (%) |
| 1 | Present Invention | 110.17 | 104.11 | 5.76 | 109.25 | 102.86 | 6.39 | 6.07 |
| 2 | Sample A | 101.81 | 91.79 | 10.02 | 100.93 | 91.65 | 9.28 | 9.65 |
| 3 | Sample B | 92.69 | 83.54 | 9.15 | 91.13 | 83.08 | 8.05 | 8.6 |
| 4 | Sample C | 110.47 | 98.56 | 11.91 | 110.2 | 99.21 | 10.99 | 11.45 |
| 5 | Sample D | 101.3 | 94.84 | 6.46 | 102.13 | 94.65 | 7.48 | 6.97 |
| 6 | Sample E | 100.99 | 94.51 | 6.48 | 100.21 | 93.51 | 6.70 | 6.59 |
| 7 | Sample F | 96.33 | 84.15 | 12.18 | 95.88 | 85.12 | 10.76 | 11.47 |
| 8 | Sample G | 104.75 | 93.19 | 11.56 | 103.25 | 93.12 | 10.13 | 10.84 |
| 9 | Sample H | 101.26 | 88.35 | 12.91 | 100.86 | 87.98 | 12.88 | 12.89 |
| 10 | Sample I | 101.58 | 87.06 | 14.52 | 100.61 | 88.01 | 12.6 | 13.56 |

TABLE 9
Oxidative degradation Analysis (%) Test,
Measured parameter: Assay (%)
Limits of measured parameter: NA
Method of measurement: HPLC Method

| | | Analysis(%) | | |
|---|---|---|---|---|
| Sr. | Name of the Products | | After | Degradation |
| No | and Details | Initial | Oxidation | in % |
| 1 | Present invention | 109.58 | 109.11 | 0.47 |
| 2 | Sample A | 101.81 | 95.63 | 6.18 |
| 3 | Sample B | 92.69 | 83.15 | 9.54 |
| 4 | Sample C | 110.47 | 101.93 | 8.54 |
| 5 | Sample D | 101.3 | 93.25 | 8.05 |
| 6 | Sample E | 100.99 | 95.47 | 5.52 |
| 7 | Sample F | 96.33 | 90.70 | 5.63 |
| 8 | Sample G | 104.75 | 96.46 | 8.29 |
| 9 | Sample H | 101.26 | 94.53 | 6.73 |
| 10 | Sample I | 101.58 | 88.92 | 12.66 |

Inference from Table 8: The assay results of Autoclave analysis (121° C. applied for 15 Minutes) indicate that the commercially available samples of Fusidic acid cream (Sr. Nos. 2-10) show more percentage drop in API content than for the product of the present invention (Sr. no. 1).

Inference from Table 9: The above Assay results of Oxidative degradation analysis (30% Hydrogen peroxide Solution over a period of 12 hours) indicate that the various Market samples of Fusidic acid cream (Sr. Nos. 2-10) show significantly higher API degradation (indicated by the percentage drop in API content) than for the product of the present invention (Sr. no. 1).

From the above data, it is evident that product of the present invention is quite stable at ambient conditions and also at elevated temperature & humid conditions of storage. Also the autoclave studies & Oxidative degradation studies further confirm the stability of the product. This is a major advantage over the currently available Fusidic acid creams. The stability of the product is further ascertained by the shelf-life prediction of the formulation using arrhenius plot of degradation employing Nova-LIMS software.

In order to prove the superiority of microbial activity of the Fusidic acid made in situ by conversion of Sodium Fusidate, experiments were carried out on commercially available creams containing Fusidic acid. Since gels use one less phase than creams, it is well known that the release of APIs from a gel are more effective than that from a cream. It is expected that the Fusidic acid in the gel of the present invention will provide similar or better results in terms of antimicrobial activity than those by a Fusidic acid cream.

The antimicrobial/antibacterial activity of the Fusidic acid made through the process of in situ conversion of sodium fusidate product is confirmed by the in vitro Antimicrobial Zone of Inhibition studies for the Fusidic acid cream produced using a similar process and tested against Staphylococcus aureus. The details of the studies are detailed below in Table 10.

TABLE 10

| | | | Zone | |
|---|---|---|---|---|
| | | | Diameter | |
| S. No | Sample | Dose | Range (mm) | Inference |
| 1 | Reference standard | 10 mcg | 21-33 | Sensitive |
| | (Fusidic acid) | 20 mcg | 20-30 | Sensitive |
| | | 50 mcg | 25-32 | Sensitive |
| 2 | Positive control (Penicillin G) | 10 Units | 21-27 | Resistant |
| 3 | Negative control (DMSO 1%) | NA | NIL | NIL |
| 4 | Sample (Test Substance) | 10 mcg | 21-23 | Sensitive |
| | (ASF-product of the present | 20 mcg | 24-26 | Sensitive |
| | invention 2%) | 50 mcg | 21-24 | Sensitive |

From the above data it is evident that Fusidic acid manufactured using a process of in situ conversion of sodium fusidate has adequate antimicrobial/antibacterial activity to treat primary and secondary bacterial infections.

The proportion of ingredients used in the manufacture of the gel of the present invention are provided in table 11.

TABLE 11

| | | | Quantity | | |
|---|---|---|---|---|---|
| | | | For | | |
| S. No | Ingredients | Specification | 350 Kg | UOM | Percentage |
| 1 | Sodium Fusidate | BP | 7.280 | Kg | 2.08% |
| 2 | Carbopol 934 P | IP | 4.375 | Kg | 1.25% |
| 3 | Triethanolamine | IP | 1.4 | Kg | 0.4% |
| 4 | Propylene Glycol | IP | 122.500 | Kg | 35% |
| 5 | Benzoic Acid | IP | 0.700 | Kg | 0.2% |
| 6 | Butylated | IP | 0.035 | Kg | 0.01% |
| | Hydroxy Toluene | | | | |
| 7 | Disodium edetate | IP | 0.035 | Kg | 0.01% |
| 8 | 1M Nitric Acid | IP | 14.000 | Lit | 4.0% |
| | Solution | | | | |
| 9 | Purified Water | IP | 199.500 | Kg | 57% |

While the above description contains much specificity, these should not be construed as limitation in the scope of the invention, but rather as an exemplification of the preferred embodiments thereof. It must be realized that modifications and variations are possible based on the disclosure given above without departing from the spirit and scope of the invention. Accordingly, the scope of the invention should be determined not by the embodiments illustrated, but by the appended claims and their legal equivalents.

1. A process to make a dermaceutical gel using sodium fusidate, said process comprising the step of using sodium fusidate as the raw active pharmaceutical ingredient and converting said sodium fusidate in situ into fusidic acid under oxygen-free environment in a gel base.

2. A process to make a dermaceutical gel using sodium fusidate as claimed in claim 1 wherein the step of using sodium fusidate as the raw active pharmaceutical ingredient and converting said sodium fusidate in situ into fusidic acid under oxygen-free environment in a gel base comprises the steps of:
a. heating water, said water being preferably purified water, in an amount between 10 to 75% w/w, more preferably 57% w/w in a mixing vessel and maintaining it at a temperature between to 50° C. to 60° C.,
b. dissolving a preservative in an amount between 0.05 to 0.5% w/w, more preferably 0.2% w/w Benzoic Acid, in the said mixing vessel,
c. adding a polymer, said polymer being preferably a natural, semisynthetic, or synthetic polymer, preferably in an amount between 1 to 5% w/w, more preferably 1.25% w/w of Carbomer 934 P to said mixing vessel of step b and thoroughly mixing using said agitator at 10 to 50 RPM and homogenizing the mixture at 1000 to 3000 RPM and under the application of vacuum of minus 1000 to minus 300 mm of mercury,
d. cooling the mixture obtained at the end of step c in said mixing vessel to 40° C. preferably by circulating cold water at a temperature between 8 to 15° C. from cooling tower in the jacket of said mixing vessel,
e. adding in an API-vessel a co-solvent, selected from a group comprising propylene glycol, hexylene glycol, polyethylene glycol-400, and the like, preferably propylene glycol, preferably in an amount between 5 to 50% w/w, more preferably 25% w/w,
f. adding sodium fusidate and dissolving using mechanical stirrer at 1000 to 3000 RPM under an inert gas flushing in the said API-vessel, said sodium fusidate being preferably in an amount between 1 to 5% w/w, more preferably 2.08%, said inert gad being preferably nitrogen,
g. adjusting the pH of the mixture obtained at the end of step f in said API-vessel to below 2 by adding acid preferably in an amount between 0.005 to 0.5% w/w, more preferably, preferably 1 molar nitric acid solution 4% w/w,
h. transferring the contents of said API-vessel obtained at the end of said step g to said mixing vessel of step d with continuous stirring at 10 to 50 RPM and homogenizing the mixture at 1000 to 3000 RPM under Nitrogen gas flushing and under application of a vacuum of minus 1000 to minus 300 mm of mercury,
i. cooling the contents of the mixing vessel of step h to 30° C. to 35° C. using circulation of chilled water from cooling tower at 8° C. to 15° C. into the jacket of said mixing vessel,
j. adjusting the pH of the contents obtained at the end of step i between 3.5 and 6.0 by adding an alkalizing agent preferably in an amount between 0.1 to 5% w/w, more preferably 0.4% w/w Triethanolamine and mixing at 10 to 50 RPM under an inert gas flushing and under vacuum of minus 1000 to minus 300 mm of mercury, said inert gas being preferably nitrogen,
k. turning off the agitator and removing the contents of the mixing vessel of step j to a storage container.

3. A process to make fusidic acid gel as claimed in claim 2 further wherein a humectant is added to the mixing vessel of said step a, said humectant being selected from a group comprising Glycerin, Sorbitol, Propylene glycol and the like, either singly or any combination thereof, to form a proportion from about 1% (w/w) to 30% (w/w), preferably 20% (w/w), more preferably 10% (w/w) of Propylene glycol.

4. A process to make fusidic acid gel as claimed in claim 3 further wherein a chelating agent is added and dissolved to the mixing vessel of said step a, said chelating agent being selected from a group comprising Disodium EDTA and the like, either singly or any combination thereof, to form a proportion from about 0.001% (w/w) to 1% (w/w), preferably 0.05% (w/w), more preferably 0.01% (w/w).

5. A process to make fusidic acid gel as claimed claim 4 further wherein an anti oxidants is added and dissolved in said step e, said anti oxidant being selected from a group comprising Butylated Hydroxy Anisole, Butylated Hydroxy Toluene and the like from about 0.001% (w/w) to 5% (w/w), preferably 0.1% (w/w), more preferably 0.01% (w/w) Butylated Hydroxy Toluene.

6. A process to make fusidic acid gel as claimed in claim 1 wherein the step of using sodium fusidate as the raw active pharmaceutical ingredient and converting said sodium fusidate in situ into fusidic acid under oxygen-free environment in a gel base comprises the steps of:
a. heating water, said water being preferably purified water, in an amount between 10 to 75% w/w, more preferably 57% w/w in a mixing vessel and maintaining it at a temperature between to 50° C. to 60° C., adding and dissolving 0.05 to 0.5% w/w preservative, more preferably 0.2% w/w Benzoic Acid, and 0.001 to 1 w/w Chelating Agent, more preferably 0.01% w/w Disodium Edetate by stiffing using agitator at 10 to 50 RPM.
b. adding to said mixing vessel, a humectant selected from a group comprising propylene glycol, hexylene glycol, polyethylene glycol-400, and the like, preferably propylene glycol, preferably in an amount between 1 to 15% w/w, more preferably 10% w/w and thoroughly mixing using an agitator at 10 to 50 RPM while maintaining the temperature of the mixture at 50° C. to 60° C.,
c. adding a polymer, said polymer being preferably a natural, semisynthetic, or synthetic polymer, preferably in an amount between 1 to 5% w/w, more preferably 1.25% w/w Carbomer 934 P to said mixing vessel of step b and thoroughly mixing using said agitator at 10 to 50 RPM and homogenizing the mixture at 1000 to 3000 RPM and under vacuum of minus 1000 to minus 300 mm of mercury,
d. cooling the mixture obtained at the end of step c in said mixing vessel to 40° C. preferably by circulating cold water at a temperature between 8 to 15° C. from cooling tower in the jacket of the mixing vessel,
e. adding in an API-vessel a co-solvent, selected from a group comprising propylene glycol, hexylene glycol, polyethylene glycol-400, and the like, preferably propylene glycol, preferably in an amount between 5 to 50% w/w, more preferably 25% w/w and adding to said API-vessel an anti-oxidant selected from a group comprising Butylated hydroxy anisole, Butylated hydroxy toluene and the like, preferably 0.01 to 0.1% w/w, more preferably 0.01% w/w of Butylated Hydroxy Toluene, and dissolving it,
f. adding sodium fusidate in said API-vessel of step e preferably in an amount of 1 to 5% w/w, more preferably 2.08% w/w, and dissolving it using mechanical stirrer at 1000 to 3000 RPM under an inert gas flushing, said inert gas being preferably nitrogen,
g. adjusting the pH of the mixture obtained at the end of step f in the API-vessel to below 2 by adding acid in an amount of 0.005 to 0.5% w/w in the form of a concentrated acid, preferably 1 Molar Nitric acid in an amount of 4% w/w,
h. transferring the contents of said API-vessel obtained at the end of step g to the mixing vessel of step d with continuous stirring at 10 to 50 RPM and homogenizing the mixture at 1000 to 3000 RPM under an inert gas flushing and under vacuum of minus 1000 to minus 300 mm of mercury, said inert gas being preferably nitrogen,
i. cooling the contents of the mixing vessel of step h to 30° C. to 35° C. using circulation of chilled water from cooling tower at 8° C. to 15° C. into the jacket of said mixing vessel,
j. adjusting the pH of the contents in mixing vessel of step 1 between 3.5 and 6.0 by adding preferably 0.1 to 5% w/w alkalizing agent, more preferably 0.4% w/w Triethanolamine and mixing at 10 to 50 RPM under an inert gas flushing and under vacuum of minus 1000 to minus 300 mm of mercury, said inert gas being preferably nitrogen,
k. turning off the agitator and removing the contents of said mixing vessel of step m to a storage container.

7. A novel dermaceutical gel containing a gel base and Fusidic acid, said Fusidic acid being made in situ under oxygen-free environment using Sodium Fusidate, wherein said cream comprises Fusidic acid made in situ by a conversion of Sodium Fusidate, and said gel base comprising a natural, semi-synthetic or synthetic polymers, a preservative, an acid, an alkali, a co-solvent, along with water, preferably purified water.

8. A novel dermaceutical gel as claimed in claim 7 wherein said gel base further comprises a buffering agent selected from a group comprising Di Sodium Hydrogen Ortho Phosphate, Sodium Hydrogen Ortho Phosphate and the like in a proportion from about 0.05% (w/w) to 1.00% (w/w).

9. A novel dermaceutical gel as claimed in claim 8 wherein said gel base further comprises an anti-oxidant selected from a group comprising Butylated Hydroxy Anisole, Butylated Hydroxy Toluene and the like in a proportion from about 0.05% (w/w) to 5% (w/w).

10. A novel dermaceutical gel as claimed in claim 9 wherein said gel base further comprises a chelating agent selected from a group comprising Disodium EDTA and the like in a proportion from about 0.05% (w/w) to 1% (w/w).

11. A novel dermaceutical gel as claimed in claim 10 wherein said gel base further comprises humectant selected from a group comprising Glycerin, Sorbitol, and the like in a proportion from about 5% (w/w) to 20% (w/w).

12. A dermaceutical gel made using the process as claimed in claim 5 the composition of which is:
from about 0.1% (w/w) to about 25% (w/w) by weight, preferably from about 0.5% to about 5% by weight and more preferably from about 1% (w/w) to 2% (w/w), of an acid form active compound, preferably 2.08% w/w of sodium fusidate and,
a gel base containing natural or semisynthetic or synthetic polymers, co-solvents, acids, alkalis, buffering agents, preservatives, anti oxidants, chelating agents, humectants, water, all weights based on the weight of the composition, wherein
natural polymers are selected from tragacanth, pectin, carrageen, agar, and alginic acid and Synthetic & semi-synthetic polymers are selected from methylcellulose, hydroxyethylcellulose, carboxymethylcellulose, hydroxypropylcellulose, hydroxypropylmethylcellulose and carbopols and the like and provided in a proportion from about 0.5% (w/w) to 10% (w/w),
co-solvents are selected from a group comprising Propylene Glycol, Hexylene Glycol, PolyEthylene Glycol-400 and the like provided in a proportion from about 5% (w/w) to 50% (w/w),
acids such as HCl, H2So4, HNO₃, Lactic acid and the like provided in a proportion from about 0.005% (w/w) to 0.5% (w/w),
an alkalizing agent, provided in the proportion of preferably 0.1 to 5% w/w, more preferably 0.4% w/w of Triethanolamine,
preservatives are selected from a group comprising Methylparaben, Propylparaben, Chlorocresol, Potassium sorbate, Benzoic acid and the like provided in a proportion from about 0.05% (w/w) to 0.5% (w/w),
buffering agents are selected from a group comprising Di Sodium Hydrogen Ortho Phosphate, Sodium Hydrogen Ortho Phosphate and the like provided in a proportion from about 0.05% (w/w) to 1.00% (w/w),
anti oxidants are selected from a group comprising Butylated Hydroxy Anisole, Butylated Hydroxy Toluene and the like provided in a proportion from about 0.05% (w/w) to 5% (w/w),
chelating agents are selected from a group comprising Disodium EDTA and the like provided in a proportion from about 0.05% (w/w) to 1% (w/w),
humectants are selected from a group comprising Glycerin, Sorbitol, and the like provided in a proportion from about 5% (w/w) to 20% (w/w).

13. A method of treatment of primary and secondary skin infections wherein the said method comprises applying a dermaceutical gel as claimed in claim 1.

14. A method of treatment of primary and secondary skin infections wherein the said method comprises applying a dermaceutical gel made using any the process as claimed in claim 2.

15. A process to make a dermaceutical gel using sodium fusidate as claimed in claim 1 wherein said oxygen-free environment is created using an inert gas.
